(19) United States
(12) Patent Application Publication
(10) Pub. No.: US 2016/0000462 A1
(43) Pub. Date: Jan. 7, 2016

(54) Trocar

(71) Applicant: Pajunk GmbH Medizintechnologie, Geisingen (DE)

(72) Inventors: Horst PAJUNK, Geisingen (DE); Heinrich PAJUNK, Geisingen (DE)

(21) Appl. No.: 14/767,209

(22) PCT Filed: Feb. 11, 2014
(86) PCT No.: PCT/EP2014/052597

§ 371 Date: Aug. 11, 2015

(30) Foreign Application Priority Data
Feb. 11, 2013 (DE) 10 2013 101 336.8

(51) Int. Cl. A61B 17/34; A61M 13/00

(52) U.S. Cl. CPC A61B 17/3474; A61B 17/3421; A61B 17/3498; A61M 13/00; A61B 19/5225

(57) ABSTRACT

A trocar with a trocar sleeve, comprising a valve device arranged at the proximal end of the trocar sleeve and comprising a trocar pin which can be inserted axially into the trocar sleeve through the valve device and which has a hollow shaft and a tapering transparent distal tip. An endoscopic optical unit, such that the adjacent body tissue can be observed through the distal tip, can be introduced into the hollow shaft of the trocar pin, and a proximal insufflation connection allows a gas to be introduced into the trocar sleeve.

The invention relates to a trocar according to the preamble of claim 1.

Trocars are used in medicine, particularly in minimally invasive surgery, in order to create a point of access into the body of a patient, e.g., into the abdominal cavity of the patient. The trocar consists of a cannula and an obturator that can be axially inserted into said cannula. The obturator is also referred to as a trocar mandrel. Sometimes, the obturator alone is also referred to as a trocar.

A valve device is arranged at the proximal end of the cannula. The valve device is used to seal in an air-tight manner the obturator inserted into the cannula or instruments and the like inserted through the cannula. Furthermore, the valve device is used to close the cannula in an air-tight manner when no obturator, instrument, or optical system is inserted. If the obturator is inserted into the cannula, the distal end of the obturator protrudes distally from the cannula. Said distal end of the obturator is designed as a tapered tip, which is used to penetrate and/or dilate the body tissue during the insertion of the trocar.

In order to reduce the risk of injuries to internal organs during the insertion of the trocar by means of the tip of the obturator, so-called optical trocars are used. In the case of these optical trocars, the obturator has a hollow shaft and at least the tapered distal tip is transparent, see-through, or at least translucent. An endoscope optical system can be inserted into the obturator, by means of which endoscope optical system the tissue lying against the outside of the tip and thus the penetration of the trocar tip can be observed through the transparent tip.

The cannula has an insufflation connection at the proximal end of the cannula. If the cannula is inserted into the abdominal wall and the obturator is pulled out of the cannula, gas can be introduced into the abdominal cavity via the insufflation connection and the cannula in order to raise the abdominal wall and expand the intracorporeal operating field. Because the insufflation cannot occur until the trocar has been inserted at least to such an extent that the distal end of the cannula is completely pushed through the abdominal wall, there remains a residual risk of injury to organs or vessels adhering to the abdominal wall even when an optical trocar is used. Therefore, an insufflation of the abdominal cavity preferably is performed by means of a so-called Veress needle before the first insertion of a trocar.

A trocar of the type mentioned at the beginning that enables insufflation through the trocar even during the first insertion of the trocar, even before the distal end of the cannula has completely penetrated the abdominal wall, is known from US 2010/0081988 A1. For this purpose, the hollow shaft of the obturator has wall bores, and gas outlet openings are provided in the distal tapered tip of the obturator. Via the proximal insufflation connection, gas is introduced into the annular space between the outer wall of the obturator and the inner wall of the cannula. Said gas enters the interior of the hollow shaft through the wall bores and can flow along the periphery of the endoscope optical system to the distal tip of the obturator, where the gas can escape through the outlet openings. The tip of the obturator is inserted through the abdominal wall under visual observation by means of the endoscope optical system. As soon as the distal tip having the gas outlet opening enters the abdominal cavity, a first insufflation can be performed via said gas outlet opening, by means of which first insufflation the abdominal wall is distanced from internal organs so that the trocar can be inserted further with minimal risk until the cannula reaches its position.

The problem addressed by the invention is that of creating a trocar of the type mentioned at the beginning that ties a simpler design.

According to the invention, said problem is solved by means of a trocar having the features of claim 1.

Advantageous embodiments of the invention are specified in the dependent claims.

The trocar according to the invention enables insufflation during the first insertion as soon as the distal tip of the obturator penetrates the abdominal wall and enters the abdominal cavity. For this purpose, gas is introduced into the cannula via the proximal insufflation connection. The gas flows in the annular space between the outer wall of the obturator and the inner wall of the cannula to the distal end of the cannula. There, the gas can escape through at least one gas-conducting channel, which extends in the jacket of a distal cylindrical end segment of the obturator in the longitudinal direction. Said at least one gas-conducting channel thus tunnels under the distal end of the cannula, which otherwise lies tightly against said cylindrical end segment. As soon as the trocar has penetrated the abdominal wall by means of the transparent distal tip of the obturator under visual observation, gas can be insufflated into the abdominal cavity via the gas-conducting channels before trocar completely enters the abdominal cavity.

The at least one gas-conducting channel must extend in the axial direction at least over the length over which the distal end of the cannula lies against the cylindrical end segment of the inserted obturator. The at least one gas-conducting channel preferably additionally extends further in the distal direction beyond said cylindrical end segment into the distally tapered tip. Thus, a first insufflation via the gas-conducting channels becomes possible as soon as the distal end of the tip has entered the abdominal cavity, i.e., still before the tapered tip has completely entered and the insertion hole has expanded to the diameter of the cylindrical end segment or the diameter of the cannula.

In an advantageous embodiment, the at least one gas-conducting channel is designed as a gas-conducting groove, which extends in the outer lateral surface-of the distal end part. The gas-conducting groove is designed as an outwardly open recessed furrow in the outer lateral surface. This embodiment offers the advantage of simple production. In another embodiment, the at least one gas-conducting channel is designed as a pipe, which extends inside the wall of the jacket of the distal end part. The pipe is closed over its entire circumference and has an inlet opening and an outlet opening only at the ends of the pipe. The pipe-shaped design of the gas-conducting channel has the advantage that the cross-section of the gas-conducting channel cannot be obstructed. However, a greater wall thickness of the jacket is required and the production of the pipe-shaped gas-conducting channels is more complex.

In a preferred embodiment, the distal tip tapered in the distal direction substantially has the shape of a cone, which has two flat areas of the lateral cone surface, which flat areas are mirror-symmetric with respect to the axial center plane. This shape of the tip makes the penetration of the tissue easier. If gas-conducting grooves are led to the distal end of the tip, these gas-conducting grooves are preferably arranged in the flat areas. During the penetration of the tip into the body tissue, the tissue lies against these flat areas with a pressure that is smaller than the pressure with which the tissue lies against the conical lateral regions of the tip. Therefore, there is a lesser tendency of the tissue to penetrate into the recessed gas-conducting grooves and to block the recessed gas-conducting grooves.

In the case of optical trocars, an endoscope optical system whose distal end surface is slanted toward the center axis of the endoscope optical system or of the obturator is often used. In particular, so-called 30° optical systems are common, in the case of which the distal end surface is slanted toward the center axis at an angle of 30°. If an endoscope optical system having a slanted distal end surface is used, the least image distortion results if the distal end surface of the endoscope optical system inserted in the obturator is directed toward the conical non-flattened lateral region of the tapered transparent tip. This optimal orientation is preferably positively effected in that the edge region of the distal end surface lying furthest in the distal direction engages in this conical peripheral region of the tip, because a circular-arc-shaped free peripheral angle is available there for the insertion of the endoscope optical system. If the endoscope optical system is inserted into the obturator, the endoscope optical system orients itself in the optimal angular position positively or possibly by means of slight rotational motions.

Additional features and advantages of the invention result from the following description of an embodiment example shown in the drawing.

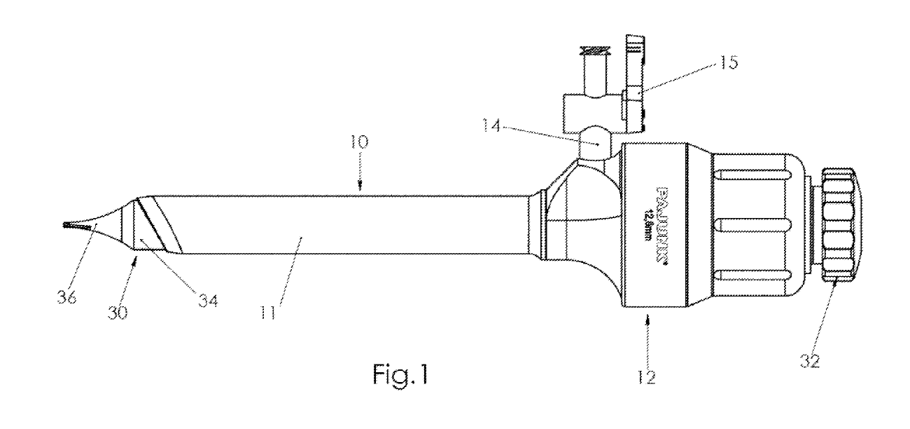

FIG. 1 shows a side view of the complete trocar.

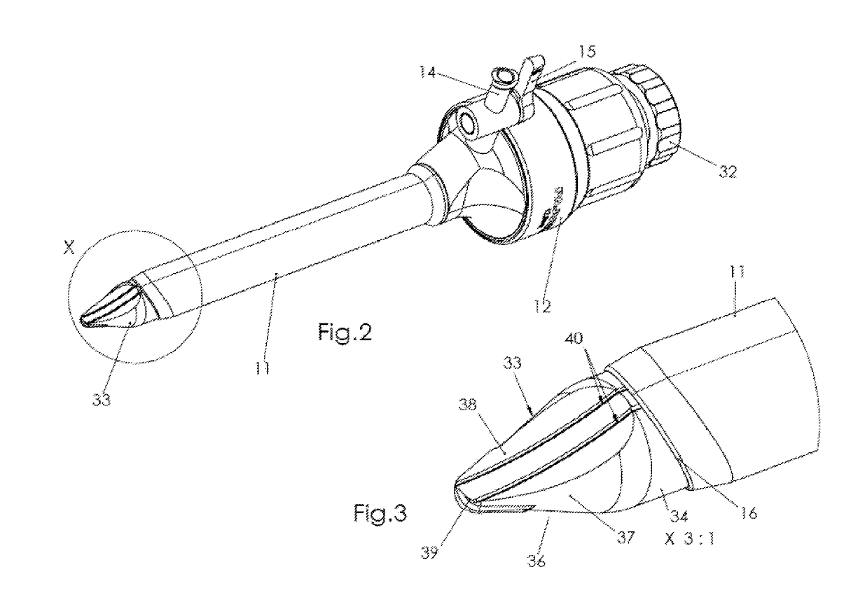

FIG. 2 shows a perspective view of said trocar.

FIG. 3 shows an enlarged illustration of the distal tip of the trocar according to image detail X in FIG. 2.

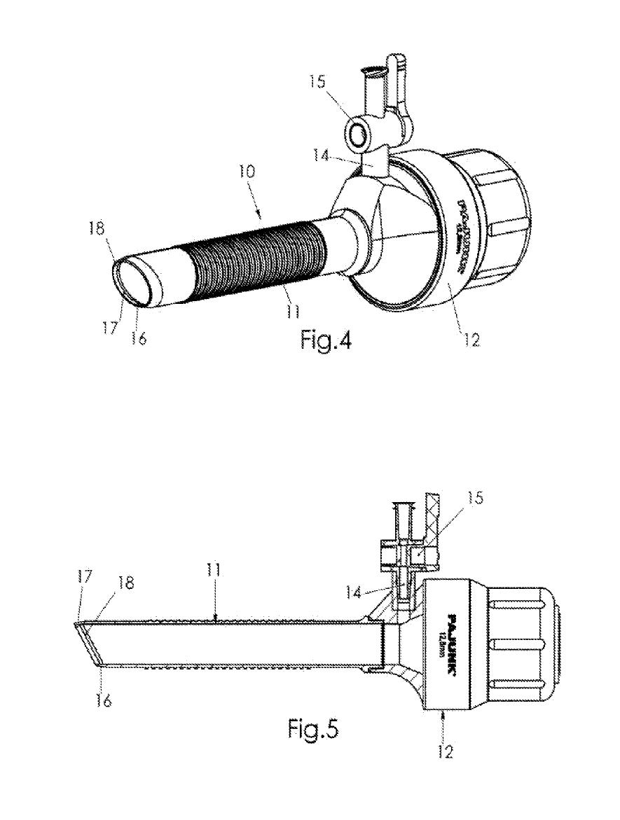

FIG. 4 shows a perspective view of the cannula.

FIG. 5 shows an axial section through the cannula.

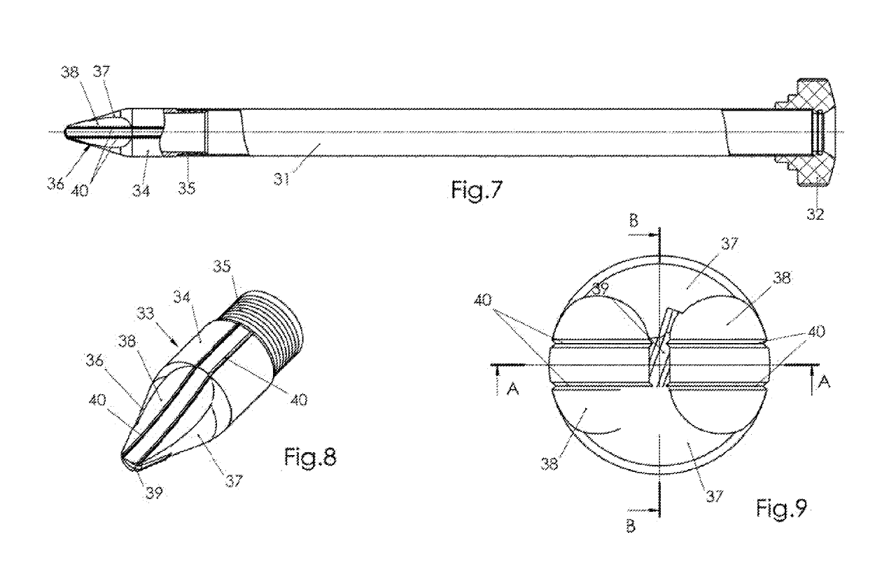

FIG. 7 shows a partially axially cut side view of the obturator.

FIG. 8 shows the distal end part of the obturator.

FIG. 9 shows an axial top view of the distal end part.

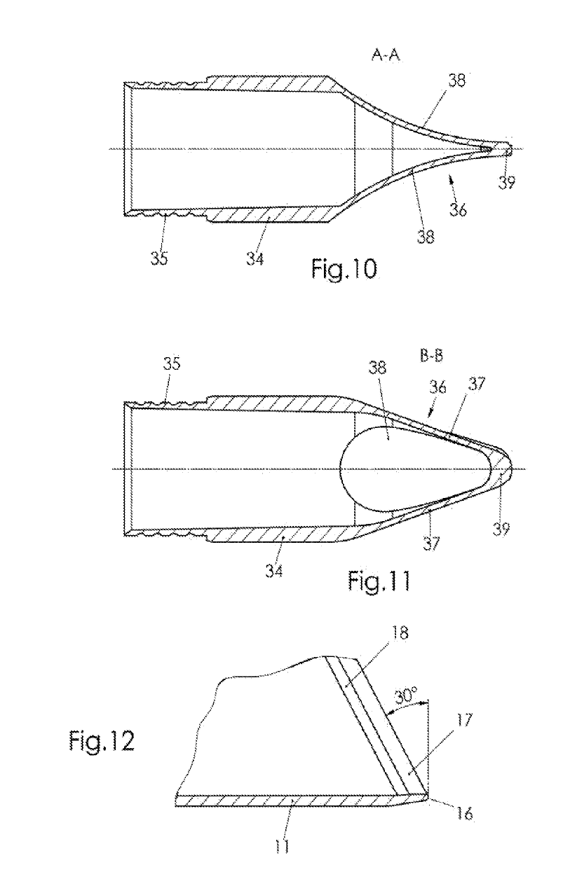

FIG. 10 shows an axial section of the distal end part according to section line A-A in FIG. 9.

FIG. 11 shows an axial section of the distal end part according to section line B-B in FIG. 9.

FIG. 12 shows an axial partial section of the distal end of the cannula.

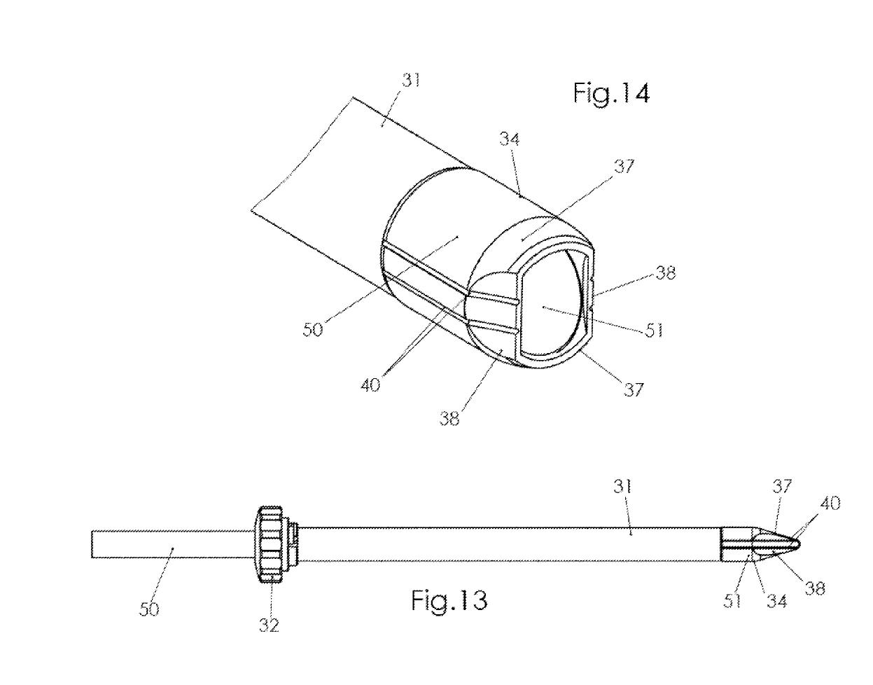

FIG. 13 shows a side view of the obturator with the endoscope optical system inserted.

FIG. 14 shows the distal end of the obturator with the endoscope optical unit inserted in perspective view.

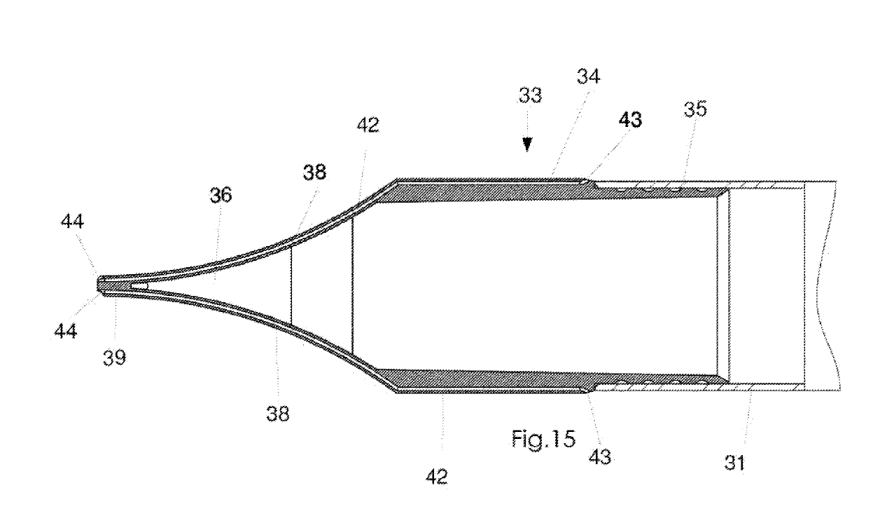

FIG. 15 shows an axial section, corresponding to FIG. 10, of the distal end part in another embodiment.

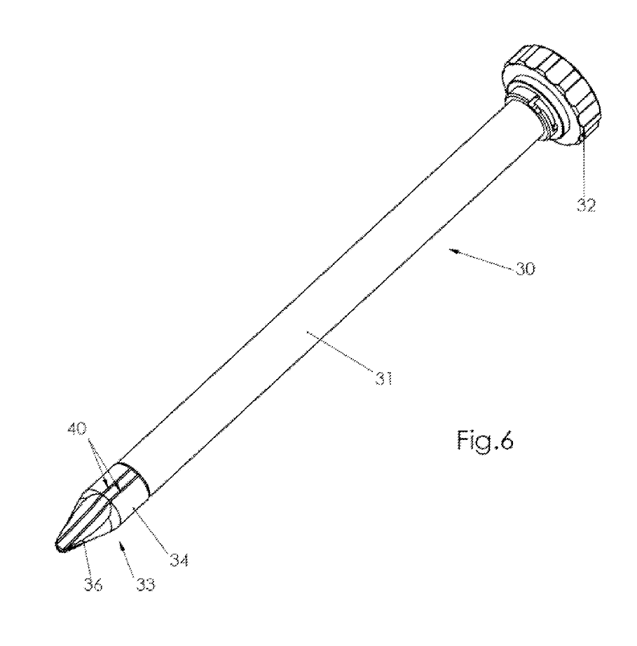

In FIGS. 1 and 2, a trocar according to the invention is shown which has a cannula 10, into which an obturator 30 can be axially inserted. The cannula 10 is shown in detail in FIGS. 4 and 5, while the obturator 30 is shown and explained in detail in FIGS. 6 to 11.

The cannula 10 consists of a cannula tube 11, which is produced, for example, from a transparent plastic. A valve device 12 is arranged at the proximal end of the cannula tube 11. The valve device 12 has a passage axially aligned with the cannula tube 11, through which passage the obturator 30 or instruments or optical systems can be inserted into the cannula tube 11. The passage of the valve device is provided with a seal, which closes the passage and thus the cannula tube 11 in an air-tight manner if no obturator or instrument is inserted through the valve device. If an obturator, an instrument, an optical system, or the like is inserted through the valve device, a second seal lies against the periphery of the obturator, of the instrument, or of the optical system in a sealing manner. Distally before the seals, an insufflation connection 14 leads radially into the passage of the valve device 12 and thus to the inner lumen of the cannula tube 11. The insufflation connection 14 can be closed by means of a tap 15. In this respect, the cannula 10 together with the valve device 12 is designed in a manner known per se.

The obturator 30 shown in FIGS. 6 and 7 has a tubular hollow shaft 31, which is preferably produced from stainless steel. A knurled knob 32, which is used to handle the obturator 30, is arranged at the proximal end of the shaft 31. A distal end part 33 is coaxially inserted into the distal end of the shaft 31 and preferably adhesively bonded to the shaft 31, as can be seen in FIG. 7.

The distal end part 33, which is shown as an individual part in FIGS. 8 to 11, is preferably produced as an injection-molded part and is composed of a transparent, preferably see-through crystal-clear plastic. The distal end part 33 has a straight, circular cylindrical end segment 34, which is coaxially inserted into the distal end of the shaft 31 by means of an attachment segment 35 and is adhesively bonded to the shaft 31. The outside diameter of the end segment 34 corresponds to the outside diameter of the shaft 31, so that the circumferential lateral surfaces of the shaft 31 and of the end segment 34 adjoin each other without a step. The distal end part 33 having a distal tip 36 tapered in the distal direction distally adjoins the end segment. 34. The distal tip 36 substantially has the shape of a cone, i.e., a right circular cone. The conical lateral surface 37 of the tip 36 is flattened on two diametrically opposite sides, so that two flat areas 38 which are mirror-symmetric with respect to a central axial plane of the end part 33 are formed, which flat areas 38 extend from the end segment 34 to the distal end of the tip. The flat areas 38 are preferably drawn inward slightly, as can be seen. particularly in FIG. 10. The distal end of the tip 36 is designed as a flat runner 39, which protrudes slightly beyond the flat areas 38 in the distal direction and is rotated toward the plane of symmetry of the two flat areas 38 at an angle about the center axis of the end part 33, as can be seen most clearly in FIG. 9. The runner 39 approximately has the shape of the working tip of a flat-head screwdriver.

At least one gas-conducting channel is formed in the jacket of the distal end part 33. The at least one gas-conducting channel has the shape of a gas-conducting groove 40 extending in the outer lateral surface of the distal end part 33. In the embodiment example shown, four gas-conducting grooves 40 are provided. The gas-conducting grooves 40 are designed as recessed furrows in the outer lateral surface. The cross-sectional shape and depth of the gas-conducting grooves 40 can be freely selected in a wide range. The gas-conducting grooves 40 have a triangular or semicircular cross-sectional profile, for example, and a depth of approximately 0.25 mm, for example. The gas-conducting grooves 40 extend in the longitudinal direction of the end part 33 and extend axially, starting from the attachment segment 35, over the entire length of the end segment 34 and extend over the distal tip 35 to the distal end of the distal tip 36, as can be seen best in FIG. 8. In the present embodiment example, pairs of two gas-conducting grooves 40 are arranged diametrically to each other. The two gas-conducting grooves 40 of each pair extend parallel to each other and are arranged in the peripheral angle of the end part 33 in such a way that she pairs of the gas-conducting grooves 40 each extend within the flat areas 38 to the distal end of the tip 36. This can be seen most clearly in FIGS. 8 and 9.

The distal end 16 of the cannula tube 11 of the cannula 10 is shown in a partial section in FIG. 12. This distal end 16 can preferably be slanted with respect to the center axis of the cannula tube 11, as FIGS. 4 and 5 show. The slant is, for example, 30°. The distal end 16 has an inner edge 17, the clear inside diameter of which corresponds to the outside diameter of the end segment 34 of the end part 33 of the obturator 30. Adjacently to this inner edge 17 in the proximal direction, the clear inside diameter of the cannula tube 10 expands in a region 18 to the somewhat larger clear inside diameter of the cannula tube 11. The clear inside diameter of the cannula tube 11 is, for example, 0.5 mm greater than the clear inside diameter of the inner edge 17.

For the use of the trocar, the obturator 30 is inserted from the proximal end through the valve device 12 into the cannula 10, until the knob 32 stops against the valve device 12. The distal tip 36 of the distal end part 33 then protrudes distally from the distal end 16 of the cannula tube 11, as is shown in FIGS. 1 to 3. At the proximal end, the obturator 30 is sealed in the valve device 12. The cannula tube 11 lies tightly against the outer circumference of the cylindrical end segment 34 of the distal end part 33 of the obturator 30 by means of the inner edge 17. Because the inside diameter of the cannula tube 11 is somewhat greater than then clear inside diameter of the inner edge 17, an annular space remains free between the outer wall of the shaft 31 of the obturator 30 and the inner wall of the cannula tube 11. The insufflation connection 14 of the valve device 12 leads into the passage of the valve device 12 and thus into this annular space between the shaft 31 of the obturator 30 and the inner wall of the cannula tube 11. This annular space is distally sealed by the inner edge 17, which lies against the circumference of the end segment 34. However, the gas-conducting grooves 40 in the end segment 34 of the distal end part 33 tunnel under the sealing of the inner edge 17, so that the gas-conducting grooves 40 form a connection between said annular space and the surroundings of the distal tip 36. If a gas or another fluid is introduced through the insufflation connection 14, said gas can enter the annular space between the cannula tube 11 and the obturator 30 via the insufflation connection 14 and can escape from the cannula tube 11 distally via the gas-conducting grooves 40.

In order to be able to insert the trocar under visual observation, an endoscope optical system 50 is inserted through the knob 32 into the obturator 30, as FIG. 13 shows. The obturator 30 is inserted into the cannula 10, as is shown in FIGS. 1 and 2. The trocar is then inserted into the abdominal wall through a skin incision, wherein the distal tip 36 of the obturator 30, together with the runner 39, causes a perforation of the tissue and a dilation of the perforation opening. Because the distal end part 33 is see-through, the body tissue in front of the distal tip 36 and the body tissue lying laterally against the distal tip 36 can be observed by means of the endoscope optical system 50. Likewise, the manner in which the tip 36 penetrates the body tissue can be observed. As soon as the distal end of the tip 36 has penetrated the abdominal wall and entered the abdominal cavity, gas can be insufflated into the abdominal cavity via the insufflation connection, the annular space within the cannula tube 11, and the gas-conducting grooves 40. The abdominal wall can thereby be distanced from internal organs of the abdominal cavity so that the further advance of the distal tip 36 into the abdominal cavity can be continued without the risk of an injury to internal organs. Because the gas-conducting grooves 40 extend within the flat areas 38 in the region of the tapered distal tip 36, the pressure of the body tissue lying against the tip 36 is absorbed substantially by the conical lateral surfaces 37 of the tip 36 and the body tissue is not pressed into the gas-conducting grooves 40, so that the gas-conducting grooves 40 remain free for the passage of gas.

An endoscope optical system known per se can be used as the endoscope optical system 50. Such an endoscope optical system 50 is often designed with a slanted distal end surface 51. In the case of so-called 30° optical systems, the distal end surface 51 is slanted at an angle of 30° toward the center axis of the endoscope optical system 50, as is shown in the embodiment example in FIGS. 13 and 14. The main viewing direction of such an endoscope optical system 50 extends perpendicularly to the end surface 51 and thus is angled at an angle of, e.g., 30° with respect to the center axis of the endoscope optical system 50 and of the obturator 30. In order to obtain an image of the body tissue at the distal tip 36 that is distorted as little as possible, it is advantageous if the endoscope optical system 50 views through a region of the distal tip 36 that is designed as a conical lateral surface 37 and that is located between the flat areas 38 in the peripheral direction. According to the invention, an endoscope optical system 50 having an end surface 51 slanted at, for example, 30° is positively oriented in this optimal viewing direction. This is effected in that the peripheral region of the end surface 51 lying furthest in the distal direction can be axially advanced further into the distal tip 36 in the distal direction if said peripheral segment lying furthest in the distal direction is located in the region of a conical lateral surface 37, as is shown in FIG. 14. In FIG. 14, the distal end of the tip 36 is cut off in order to make the orientation of the end surface 51 of the endoscope optical system 50 clearly visible. By slightly rotating the endoscope optical system 50 during the insertion, the optimal angular orientation of the end surface 51 within the distal tip 36 results. If the distal end surface 51 has entered this one conical surface lateral surface 37 of the distal tip 36 of the obturator 30, the viewing direction of the end surface 51 is directed toward the diametrically opposite conical lateral surface 37, which delivers the optical image with the least distortion.

In a further embodiment shown in FIG. 15, the at least one gas-conducting channel is designed as a gas-conducting pipe 42. In the embodiment example shown, two gas-conducting pipes 42 arranged diametrically to each other are provided. The gas-conducting pipes are designed as pipes which are completely embedded in the wall of the end part 33 and which are closed over their entire circumference. The gas-conducting pipe 42 has an inlet opening 43 only at the proximal end of the cylindrical end segment 34 and a distal outlet opening 44. The at least one gas-conducting pipe 42 extends axially at least over the length of the cylindrical end segment 34, against which the distal end 16 of the cannula 10 lies. The at least one gas-conducting pipe 42 preferably extends distally beyond the cylindrical end segment 34 into the distal tip 36 so that the outlet openings 44 lie at the front distal end of said tip 36.

In this second embodiment, the distal end part 33 is preferably produced from plastic in axially separate partial shells, wherein the gas-conducting pipes 42 are designed as furrows in the abutting surfaces by means of which the partial shells are joined.

The use of the trocar having the distal end part 33 of the obturator 30 in this second embodiment corresponds completely to the previously described use in the first embodiment.

### LIST OF REFERENCE SIGNS

10 Cannula

11 Cannula tube

12 Valve device

14 insufflation connection

15 Tap

16 Distal end

17 Inner edge

18 Region

30 Obturator

31 Shaft

32 Knob

33 Distal end part

34 End segment

35 Attachment segment

36 Distal tip

37 Conical lateral surface

38 Flat areas

39 Runner

40 Gas-conducting groove

42 Gas-conducting pipe

43 Inlet opening

44 Outlet opening

50 Endoscope optical system

51 End surface

1. A trocar, comprising:
a cannula;
a valve device arranged at the proximal end of the cannula; and an obturator that can be inserted axially into the cannula through the valve device, which obturator has a hollow shaft and a transparent tapered distal tip, into which hollow shaft of the obturator an endoscope optical system can be inserted such that the contacting body tissue can be observed through the distal tip, a proximal insufflation connection making it possible to introduce a gas into the cannula, the distal end of the cannula lying against the outer circumference of a cylindrical end segment of the obturator if the obturator is inserted into the cannula, which cylindrical end segment proximally adjoins the tapered tip, and the gas introduced through the insufflation connection being able to enter an annular space between the outer wall of the obturator and the inner wall of the cannula and to escape via a distal gas outlet if the obturator is inserted,
wherein the distal gas outlet is formed by at least one gas-conducting channel, which extends in the jacket of the cylindrical end segment in the longitudinal direction of the obturator and extends axially at least over the length over which the distal end of the cannula lies against the cylindrical end segment of the inserted obturator.

2. The trocar according to claim 1, wherein the at least one gas-conducting channel extends distally beyond the cylindrical end segment into the jacket of the tapered distal tip.

3. The trocar according to claim 2, wherein the at least one gas-conducting channel is led to the distal end of the tip.

4. The trocar according to claim 1, wherein at least two gas-conducting channels are provided, which are arranged diametrically to each other.

5. The trocar according to claim 1, wherein the at least one gas-conducting channel is formed by at least one gas-conducting groove, which extends as a recessed furrow in the outer lateral surface of the cylindrical end segment and possibly of the tapered distal tip.

6. The trocar according to claim 1, wherein the at least one gas-conducting channel is formed by a gas-conducting pipe, which extends in the wall of the jacket of the cylindrical end segment and possibly of the tapered distal tip.

7. The trocar according to claim 1, wherein the tapered distal tip substantially has the shape of a cone having two flat areas which are mirror-symmetric with respect to the axial center plane.

8. The trocar according claim 2, wherein the gas-conducting grooves extend in the flat areas.

9. The trocar according to claim 1, wherein the tapered distal tip and the cylindrical end segment adjoining the tapered distal tip form an injection-molded part composed of a transparent plastic.

10. The trocar according to claim 7, wherein the endoscope optical system has a distal end surface slanted toward the center axis, the edge of which distal end surface lying furthest in the distal direction engages in the peripheral region of the distal tip having the conical lateral surface between the flat areas when the endoscope optical system is inserted into the obturator.

11. The trocar according to claim 10, wherein the endoscope optical system has a distal end surface slanted at 30°.
